(19) United States
(12) Patent Application Publication
(10) Pub. No.: US 2012/0251628 A1
(43) Pub. Date: Oct. 4, 2012

(54) COMPOSITIONS AND METHODS FOR TREATMENT OF CANCER

(76) Inventor: Young Joo MIN, Ulsan (KR)

(21) Appl. No.: 13/436,141

(22) Filed: Mar. 30, 2012

Related U.S. Application Data
(60) Provisional application No. 61/469,415, filed on Mar. 30, 2011.

(51) Int. Cl. A61K 33/36; A61P 35/00

(52) U.S. Cl. 424/623

(57) ABSTRACT

Compositions and methods for treating solid tumors are provided. Intraperitoneal administration of sodium meta arsenite, alone or in combination with an anti-cancer agent, results in tumor necrosis and delayed tumor growth. A particularly effective treatment for colon and rectal cancer is a combination of sodium meta arsenite administered intraperitoneally and irinotecan.

### CROSS-REFERENCE TO RELATED APPLICATION

This application claims priority from U.S. provisional patent application Ser. No. 61/469,415, filed Mar. 30, 2011.

### FIELD OF THE INVENTION

This invention relates to the treatment of solid tumors by intraperitoneal injection of sodium meta arsenite, alone or together with a second anti-cancer agent. More particularly, the invention relates to the treatment of solid tumors using a combination therapy of intraperitoneal administration of sodium meta arsenite and administration of an anti-cancer agent having a different mechanism of action from intraperitoneally administered sodium meta arsenite, e.g., is not a vascular disrupting agent.

### BACKGROUND OF THE INVENTION

Sodium meta arsenite is an anticancer agent that previously has been formulated for oral administration. Clinical studies have shown that sodium meta arsenite has anticancer effect against prostate cancer and currently, a number of clinical studies of sodium meta arsenite for treatment of a wide variety of other types of cancer are being conducted in Korea. It has been reported that sodium meta arsenite destroys and controls cancer cells by causing DNA damage only at the telomeres of chromosomes in cancer cells (Phatak P, et al., 2008, Clinical Cancer Research). However, these clinical results do not fully explain the mechanism of action of sodium meta arsenite, which exhibits a broad band of effectiveness against a variety of cancer types and is significantly less toxic than most anticancer agents.

Arsenic (As) was shown to have clinical effects in patients with acute promyelocytic leukemia (APL) in China in the 1970s. Since then, more than 80 percent of patients to which arsenic trioxide (ATO) was administered have shown therapeutic effects without acute toxicity. The anti-cancer activity of ATO has been attributed in the scientific literature as due to a variety of mechanisms including an anti-tubulin effect, differentiation induction, apoptosis, anti-proliferative activity and angiogenesis inhibition. Young S. Lew et al. (1999. Cancer Research) observed acute tumor vascular shutdown and massive tumor necrosis similar to that observed in a new class of drugs referred to as ‘vascular disrupting agents’ (VDAs) when ATO was administered to a murine tumor model.

Several, anti-angiogenic drugs that have been tested in clinical trials as treatments for solid tumors, e.g., lung cancer and colorectal cancer, have been shown to normalize the pathological abnormalities of tumor vessels and control neovascularization. From these clinical trials, a class of therapeutic agents known as vascular disrupting agents (VDAs) and a new approach to treating cancer have emerged. VDAs are agents with a different mechanism of action from known anti-VEGF agents, such as bevacizumab, sunitinib and sorafenib. When combined with tubulin in the endothelial cells of blood vessels, VDAs inhibit blood flow to tumors and cause necrosis of tumors by blocking tumor blood vessels within a few hours after administration. (Thorpe P E, 2004, Clinical Cancer Research). Because VDAs rarely affect normal blood vessels, they do not cause malfunction of liver, kidneys, brain or any other normal organs that are often seen as a result of blood vessel occlusion. Therefore, VDAs have been reported to be safe anti-cancer agents which exhibit low toxicity (Hinnen P, et al., 2007, British Journal of Cancer).

The selective destruction of tumor blood vessels by VDAs appears to take advantage of the pathological abnormalities of tumor blood vessels. VDAs selectively bind to the tubulin of endothelial cells in tumor blood vessels and cause cell-to-cell junction or cytoskeleton disruption, transformation of endothelial cell shape, an increase in protein permeability in the blood tumor blood vessels and vascular occlusion due to vascular constriction arising from increased interstitial pressure, increased blood viscosity, hemo-concentration, serotonin secretion, etc. (Hinnen. P, et al., 2007, British Journal of cancer). This causes tumor hypoxia and necrosis. VDAs under development or currently in clinical trials include ASA404, CA4DP, AVE8062A, OXi4503, etc. (Keespies A, et al., 2005, Neoplasia). In Phase II clinical trials, ASA404 has been shown to enhance survival of advanced lung cancer patients in clinical trials when used in combination with the standard anti-cancer therapy. (McKeage M J, et al., 2008, British Journal of Cancer).

There is a need for anti-cancer drugs and combinations of drugs that selectively target tumor cells, while leaving normal cells intact and which have low toxicity. The present invention addresses that need.

### SUMMARY OF THE INVENTION

In one aspect of the invention there is provided a method of treating a solid tumor in a patient in need thereof comprising intraperitonealy administering to the patient a therapeutically effective amount of each of sodium meta arsenite and an anticancer agent that is not a vascular disrupting agent. In certain embodiments of this aspect of the invention, the solid tumor is a colon cancer-associated tumor and the anticancer agent is irinotecan. In other embodiments the patient is suffering from rectal cancer. In certain embodiments of this aspect of the invention, the sodium meta arsenite is administered at weekly intervals. In certain embodiments of this aspect of the invention, any anti-cancer agent having a different mechanism of action from intraperitoneally administered sodium meta arsenite is administered to the patient.

### BRIEF DESCRIPTION OF THE DRAWINGS

FIG. 1 is a series of photographs of tumor tissue before and after treatment with control or sodium meta arsenite (10/mg/kg).

FIG. 2A-2D are photographs of hematoxylin and eosin stained tumor tissue sections. 2A is tissue from the control group; 2B is tumor tissue at 8 hours after treatment with sodium meta arsenite (10/mg/kg); 2C is tumor tissue at 24 hours after treatment with sodium meta arsenite (10/mg/kg); and 2D is tumor tissue at 48 hours after treatment with sodium meta arsenite (10/mg/kg). V: non-necrotic tissue; N; necrotic tissue.

FIG. 3A is a bar graph showing percent cytotoxicity of sodium meta arsenite over time (10 mg/kg) in HUVECs. FIG. 3B is an analysis of apoptosis of HUVECS by sodium meta arsenite at 10 μM, 20 μM and 50 μM after 24 and 48 hours exposure to 10 mg/kg sodium meta arsenite.

FIG. 4 shows a portion of a Western blot showing the quantitative change in microtubules of HUVECs treated with sodium meta arsenite. S: isolated tubulin; P: polymerized tubulin

FIG. 5 is a portion of a blot of an SDS-polyacrylamide gel showing the quantitative change in tubulin from HUVECs treated with sodium meta arsenite at various concentrations.

FIG. 6 shows sections of a 1.5% agarose gel showing quantitative changes in tubulin mRNA obtained from HUVECs after treatment with sodium meta arsenite at various concentrations. GAPDH is glyceraldehyde-3-phosphate dehydrogenase mRNA (control).

FIG. 7 shows micrographs of HUVECs exposed to sodium meta arsenite at various concentrations for 24 or 48 hours. The micrographs show the change in microtubules.

FIG. 8A-8D are graphs showing relative tumor volume after a period of treatment with irinotecan and/or sodium meta arsenite in a mouse allograft model of colon cancer. A. sodium meta arsenite was injected 24 hours after the initial Irinotecan injection; B. sodium meta arsenite was injected 24 hours and 72 hours after Irinotecan; C. sodium meta arsenite was injected 72 hours after Irinotecan; and sodium meta arsenite was injected 30 minutes after Irinotecan was injected. The key to the symbols for each of the graphs is shown in 8D.

FIG. 9A-9C are graphs showing quantitative changes in Kep values in a Ct6 colon cancer xenograph mouse model in animals treated with (A) saline (control); (b) ATO; and (C) KML001.

FIG. 10A-10B show a graph (10A) showing quantitative changes in Kep values of pre- and post-treatment animals treated with ATO, KML001 or saline (control) (* p<0.05) and a table (10B) showing average Kep values of each group.

In each of the drawings KML001 is sodium meta arsenite; the terms are used interchangeably throughout the specification.

### DETAILED DESCRIPTION OF THE INVENTION

The inventor has discovered that sodium meta arsenite acts in vivo as a vascular disrupting agent when administered via intraperitoneal injection. At therapeutic concentrations, sodium meta arsenite selectively targets cancer cells and is effective against a broad range of cancers. VDAs, including sodium meta arsenite, may be applied to almost all solid tumors without limit because they selectively target tumor blood vessels, rather than the tumors themselves. VDAs, like sodium meta arsenite, have few side effects.

Sodium meta arsenite has been shown herein to exhibit vascular disrupting effects in Xenograft mouse models of tumors, but does not have any effect on the blood supply to normal organs such as liver and kidney. Because it does not affect the supply of blood to normal organs, the vascular disruption activity of sodium meta arsenite appears to be limited to tumor blood vessels. Based on results of studies using human umbilical vein endothelial cells (HUVECs), it appears that the vascular disrupting effect of intraperitoneally administered sodium meta arsenite results from morphologial changes of endothelial cells caused by cytoskeleton-associated protein degradation of tubulin. Thus, intraperitoneal injection of sodium meta arsenite provides a new vascular disrupting agent.

The inventor has also discovered that the therapeutic effects of sodium meta arsenite are complemented, and the anti-cancer effects enhanced, by combinations of intraperitoneally administered sodium meta arsenite with administration of other, conventional anti-cancer agents, such as anti-VEGF drugs or other drugs or treatments, particularly those drugs and treatments having a different mechanism of action from intraperitoneally administered sodium meta arsenite, including sodium meta arsenite when orally administered. However, the therapeutic effects of sodium meta arsenite may also be enhanced by combinations of intraperitoneally administered sodium meta arsenite with administration of another vascular disrupting agent. The therapeutic effects of such combinations enable treatment of large tumors, for example, which are particularly difficult to treat because most anti-cancer drugs and radiation primarily affect the periphery of tumors, leaving the central portion viable. VDAs, including intraperitoneally administered sodium meta arsenite, cause necrosis of the central portion of tumors, while non-VDA anti-cancer agents and radiation therapy generally cause apoptosis of cancer cells mainly in the peripheral part of tumors.

A particularly effective combination therapy is the combination of intraperitoneal injection(s) of sodium meta arsenite and irinotecan. Intermittent intraperitonal injection of sodium meta arsenite, such as for example, once per week, or every other day, in combination with another anti-cancer agent having a different mechanism of action is also particularly effective in the treatment of solid tumors. As described above, intraperitoneal injection of sodium meta arsenite may be used in combination with any anti-cancer agent, such as radiation, VEGF-inhibitors, oral dosage forms of sodium meta arsenite, VDAs and the like. Although the invention is described herein in detail with regards to the particular combination of sodium meta arsenite (intraperitoneally administered) in combination with irinotecan for the treatment of colon or rectal cancer, the same methods and treatment regimens, dosages and other details of treatment as applied to this particular combination and of agents and specific cancers may be applied to other combinations of intraperitoneal sodium meta arsenite and anti-cancer agent for the treatment of any solid tumor.

The combination of intraperitonally administered sodium meta arsenite and administration of irinotecan by any acceptable route, including intraperitoneally for example, is particularly effective in the treatment of colon cancer and rectal cancer. Sodium meta arsenite administered in this fashion acts as a VDA and enhances the previously observed anti-cancer effects of irinotecan in the treatment of colon cancer.

In one aspect of the invention there are provided methods for treating cancer, such as solid tumors, comprising intraperitoneally administering a therapeutically effective amount of sodium meta arsenite and a therapeutically effective amount of irinotecan to a patient in need, e.g., a patient suffering from colon cancer or rectal cancer. In certain embodiments of the invention, the invention provides a method for preventing, treating, and/or managing primary or metastatic colon cancer or rectal cancer, the method comprising intraperitoneally administering to a subject in need thereof a prophylactically or therapeutically effective amount or regimen of sodium meta arsenite and administering irinotecan via any effective route. In one embodiment, the sodium meta arsenite is administered once per week or less, as needed. In certain embodiments, a reduction in size, necrosis or cancer cell number is monitored periodically. Accordingly, in a specific embodiment, the invention provides a method of preventing, treating and/or managing colon or rectal cancer in a subject, the method comprising: (a) intraperitoneally administering to a subject in need thereof one or more doses of an effective amount of sodium meta arsenite and administering one or more doses of an effective amount of irinotecan; (b) monitoring the colon and/or rectal tumor(s) in the subject prior to, during, and/or after administration of a predetermined number of doses of one or both drugs and prior to the administration of a subsequent dose of one or both drugs; and (c) detecting a reduction in size or necrosis of the colon and/or rectal cancer associated tumor(s) in the subject and repeating step (a) and/or step (b) as necessary. In certain aspects of the invention, the sodium meta arsenite is administered intermittently, such as weekly.

In certain embodiments, the amount or regimen of sodium meta arsenite and irinotecan results in at least a 1.1-, 1.2-, 2-, 3-, 4-, 5-, 10-, 25-, 50-, 75-, 100-, 200- or 1000-fold reduction in size of the colon/rectal cancer-associated tumor(s). In some embodiments, the reduction in size of the tumor(s) results after two weeks, a month, two months, three months, four months, six months, nine months, 1 year, 2 years, 3 years, or 4 years of administration of the regimen.

In some embodiments, the amount or regimen of sodium meta arsenite and irinotecan results in a reduction in the bulk tumor size, necrosis of the tumor, and/or a reduction in the amount of colon and/or rectal cancer cells. In certain embodiments, the reduction in the bulk tumor size, necrosis and the reduction in the amount of colon and/or rectal cancer cells are monitored periodically. Accordingly, in one embodiment, the invention provides a method of preventing, treating and/or managing colon and/or rectal tumor(s) in a subject, the method comprising: (a) intraperitoneally administering to a subject in need thereof one or more doses of a therapeutically effective amount of sodium meta arsenite and administering one or more doses of irinotecan; (b) monitoring the colon and/or rectal cancer cells and the bulk colon and/or rectal cancer tumor size and/or integrity in the subject prior to, during, and/or after administration of a certain number of doses of one or both drugs and prior to the administration of a subsequent dose of one or both drugs; and (c) detecting a reduction in the amount of colon and/or rectal cancer cells and/or the bulk colon tumor size and/or tumor necrosis in the subject by repeating step (a) as necessary.

In some embodiments, the reductions in the cancer cells and the bulk cancer-associated tumor size or integrity result after two weeks, a month, two months, three months, four months, six months, nine months, 1 year, 2 years, 3 years, 4 years, 5 years or 10 years of administration of the regimen.

A number of known methods can be used to assess the bulk size of the tumor. Non-limiting examples of such methods include imaging methods (e.g., computed tomography (CT), magnetic resonance imaging (MRI), ultrasound, X-ray imaging, PET scans, radionuclide scans, bone scans), visual methods (e.g., through surgery), blood or biopsy tests (e.g., detection of EGFRvIII, glioblastoma cells often contain this mutation), histopathology, cytology, and flow cytometry.

In some embodiments, the bulk tumor size and/or integrity can be measured by assessments based on imaging methods. In specific embodiments, the assessments are performed in accordance with the Response Evaluation Criteria In Solid Tumors (RECIST) Guidelines, which are set forth in Therasse, P. et al., “New Guidelines to Evaluate the Response to Treatment in Solid Tumors,” J. of the Nat. Canc. Inst. 92(3), 205-216 (2000). For instance, in specific embodiments, lesions in the subject that are representative of bulk tumor size are selected so that they are at least ≧20 mm in their longest diameter at baseline (prior to treatment) when conventional imaging techniques are used (e.g., conventional CT scan, PET scan, bone scan, MRI or x-ray) and lesions that are at least ≧10 mm in their longest diameter at baseline should be selected when spiral CT scanning is used.

The invention provides a method of preventing, reducing, treating, or eliminating tumors (primary and metastasized), such as colon or rectal cancer tumors, the method comprising administering to a subject in need thereof a therapeutically and/or prophylactically effective amount or regimen of sodium meta arsenite and irinotecan, the method comprising administering sodium meta arsenite and irinotecan to the subject at doses equal to or less than the maximum tolerated dose (MTD) for each drug or equal to or less than the no observed adverse effect level (NOAEL) for each drug. The MTDs of sodium meta arsenite and irinotecan are typically based on the results of Phase I dose escalation trials.

The NOAEL, as determined in animal studies, is often used to determine the maximum recommended starting dose for human clinical trials. The NOAELs can be extrapolated to determine human equivalent dosages (HEDs). Typically, such extrapolations between species are conducted based on the doses that are normalized to body surface area (i.e., mg/m²). In specific embodiments, the NOAELs are determined in either mice, hamsters, rats, ferrets, guinea pigs, rabbits, dogs, primates, primates (monkeys, marmosets, squirrel monkeys, baboons), micropigs and minipigs. For a discussion on the use of NOAELs and their extrapolation to determine human equivalent doses, see Guidance for Industry Estimating the Maximum Safe Starting Dose in Initial Clinical Trials for Therapeutics in Adult Healthy Volunteers, U.S. Department of Health and Human Services Food and Drug Administration Center for Drug Evaluation and Research (CDER), Pharmacology and Toxicology, July 2005. Accordingly, in certain embodiments, the regimen comprises administering a therapy at a dose less than the HED. For instance, the invention provides a method of preventing recurrence of colon tumors in a subject in remission, the method comprising administering to a subject in need thereof a prophylactically or therapeutically effective amount or regimen of sodium meta arsenite and irinotecan, the method comprising administering sodium meta arsenite to the subject at dose equal to or less than the HED.

In one embodiment, the dosage of each of sodium meta arsenite and irontecan (or other anti-cancer drug, as the case may be) administered to a subject to prevent, treat, eliminate, and/or manage colon/rectal cancer in a subject is 500 mg/kg or less, preferably 250 mg/kg or less, 100 mg/kg or less, 95 mg/kg or less, 90 mg/kg or less, 85 mg/kg or less, 80 mg/kg or less, 75 mg/kg or less, 70 mg/kg or less, 65 mg/kg or less, 60 mg/kg or less, 55 mg/kg or less, 50 mg/kg or less, 45 mg/kg or less, 40 mg/kg or less, 35 mg/kg or less, 30 mg/kg or less, 25 mg/kg or less, 20 mg/kg or less, 15 mg/kg or less, 10 mg/kg or less, 5 mg/kg or less, 2.5 mg/kg or less, 2 mg/kg or less, 1.5 mg/kg or less, or 1 mg/kg or less of a patient's body weight. The dosage may be administered as a single dosage of each drug or as multiple dosages or as a single dose of one drug and multiple doses of the other throughout the day. In certain embodiments, from about 0.001 mg/kg to about 20 mg/kg of sodium meta arsenite is administered, and in other embodiments, sodium meta arsenite is administered at a dosage amount of about 0.01 to 20 mg. In other embodiments, about 0.01 to 50 mg sodium meta arsenite is administered on an intermittent schedule, e.g., every other day, weekly, biweekly. Sodium meta arsenite may also administered in equal increments multiple times throughout the day to meet the desired dosage amount. Irinotecan (or other anti-cancer agent used in combination with sodium meta arsenite according to the invention) may be administered as needed. For example, irinotecan or other drug may be administered on a different schedule than sodium meta arsenite, via a different administration route, in different dosage amounts, and may also be administered together with sodium meta arsenite, e.g., at the same time, same schedule, or via the same administration route.

In one embodiment the initial dose of irinotecan and/or sodium meta arsenite may be higher or lower than subsequent doses. The dosage amount of each of irinotecan and sodium meta arsenite may be adjusted as necessary. For example, an initial dose of irinotecan may be 150 mg/m² and may be adjusted to as low as 50 mg/m² in 25 to 50 mg increments depending on individual patient tolerance to treatment. Provided intolerable toxicity does not develop, treatment with additional courses of therapy may be continued indefinitely or as needed. The frequency of administration of sodium meta arsenite and other anti-cancer drug of therapy depends on several factors including the health of the patient and stability of the disease.

In certain embodiments, the dosage of sodium meta arsenite and irinotecan administered to a subject to prevent, treat, eliminate, and/or manage colon cancer in a subject is a unit dose of each drug of independently selected from 0.001 mg to 2.5 mg, such as for example (independently for each drug) 0.001 mg to 20 mg, 0.01 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7 mg, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg, daily or less frequently. In certain embodiments the daily dosage of sodium meta arsenite administered to a patient is about 2.5 to 15 mg/day or less frequently, such as weekly. The amount of each drug is adjusted to the needs of the patient. The dosage may be increased or decreased during the treatment period, taking into account the patient's overall health and response.

In certain embodiments, the daily dosage of sodium meta arsenite and dosage of irinotecan administered to a subject to prevent, treat, eliminate, and/or manage colon cancer in a subject is in the range of 0.01 to 10 g/m², and more typically, in the range of 0.1 g/m m² to 7.5 g/m², of the subject's body weight. In one embodiment, the dosage administered to a subject is in the range of 0.5 g/m² to 5 g/m², or 1 g/m² to 5 g/m² of the subject's body's surface area for each drug.

In certain embodiments of the invention, a daily dosage of sodium meta arsenite and irinotecan are both administered to the patient on consecutive days, such as for three to twenty one consecutive days, although the total number of days of treatment for each drug may vary from patient to patient. In other embodiments, sodium meta arsenite and irinotecan are both administered for a period of time, e.g., three days, followed by a period of time in which the patient is not treated with sodium meta arsenite, e.g., three days, although it is not necessary that the same dosing schedule be used for both drugs. In other embodiments, sodium meta arsenite and irinotecan may be administered at the same time or at different times during the same day or may be administered on different days. Treatment may be repeated using the same pattern of treatment or a different pattern of treatment. In other embodiments, the patient may be treated with another anti-cancer agent, such as radiation therapy or chemotherapy during the periods when sodium meta arsenite is not administered; in those embodiments, the patient does not necessarily receive treatment every day. In certain other embodiments, the dosage of irinotecan is in the range of from 125 mg/m² to 180 mg/m² administered once a week as necessary, for example, for up to four weeks.

In certain embodiments, sodium meta arsenite is administered to the patient within 24 hours to 72 hours of administration of a therapeutically effective dosage amount of irinotecan (or any other applicable treatment or anti-cancer drug). This administration regiment results in significant tumor growth delay and tumor necrosis.

Treatment with sodium meta arsenite and irinotecan (or other anti-cancer agent) may be carried as long as necessary to reduce or eliminate the tumor(s). Treatment may be as short as three days, for example and may continue for up to six months or longer. For example, treatment may be carried out for three days, such as three consecutive days, and up to three months or longer, although during the longer period the patient need not necessarily receive treatment every day. In one embodiement, sodium meta arsenite is administered intraperitoneallly once per week. Treatment with one drug (sodium meta arsenite or irnotecan or other drug or anti-cancer agent) may be carried out for a longer period than treatment with the other drug or agent and/or may be more frequent than with the other drug or agent.

In some embodiments, the prophylactically and/or therapeutically effective amount or regimen of sodium meta arsenite and irnotecan or other drug is administered in combination with one or more additional therapies, such as radiation treatment, proteosome inhibitors and/or with chemosensitizers. In certain embodiments, the dosages of the one or more additional therapies used in the combination therapy is lower than those which have been or are currently being used to prevent, treat, and/or manage the cancer being treated. The recommended dosages of the one or more additional therapies currently used for the prevention, treatment, and/or management of the particular cancer can be obtained from any reference in the art including, but not limited to, in the case of colon cancer, Hardman et al., eds., Goodman & Gilman's The Pharmacological Basis Of Basis Of Therapeutics, 10th ed, Mc-Graw-Hill, New York, 2001; Physician's Desk Reference (60.sup.th ed., 2006), which are incorporated herein by reference in their entirety.

The combination therapy can be administered in cycles such as where a therapeutic agent is administered on day one, followed by a second on day two, then a period without administration, followed by re-administration of the therapeutics on different successive days, is comprehended within the present invention. The dosage regimen will be determined by the patient's physician taking into account such factors as the patient's overall health, age, weight, response to treatment, and other relevant factors.

Sodium meta arsenite and irinotecan (or other or additional therapy) can be administered separately, simultaneously, or sequentially. The combination of agents may be administered to a subject by the same or different routes of administration. In alternative embodiments, sodium meta arsenite and irinotecan are administered in a single composition for intraperitoneal injection. The combination therapy can also be administered in cycles such as where one drug (sodium meta arsenite or irinotecan) is administered on day one, followed by the other drug on day two, then a period without administration, followed by re-administration of the therapeutics on different successive days.

Kits containing dosage units of sodium meta arsenite formulated for intraperitoneal injection and irinotecan or other drug, formulated for oral, intraperitoneal or intravenous administration for example, are contemplated by the invention. The kits may contain a chemosensitizing agent, if appropriate. The kit may contain sufficient amounts of the treatment agents for one or several rounds of treatment.

Pharmaceutical compositions and dosage forms of each of sodium meta arsenite and irinotecan or other drug comprise the drug, solvate, hydrate, stereoisomer, clathrate, or prodrug thereof. Pharmaceutical compositions and dosage forms of the invention can further comprise one or more excipients.

Single unit dosage forms of the drugs (other than sodium meta arsenite) of the invention are suitable for oral and parenteral (e.g., subcutaneous, intravenous, bolus injection, intramuscular, or intraarterial), transdermal or transcutaneous administration to a subject, while sodium meta arsenite is formulated for intraperitoneal injection. Examples of dosage forms include, but are not limited to: tablets; caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a subject. In certain embodiments of the invention, sodium meta arsenite is administered intraperitoneally and irinotecan is administered intravenously.

The composition, shape, and type of dosage forms of the invention will typically vary depending on their use. For example, dosage forms used in the treatment of an aggressive colon cancer may contain larger amounts of sodium meta arsenite and/or irinotecan than a dosage forms used in the treatment of a less aggressive colon tumor. Similarly, a parenteral dosage form may contain smaller amounts of irinotecan than an oral dosage form used to treat the same disease. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990), incorporated herein by reference.

Typical pharmaceutical compositions and dosage forms of sodium meta arsenite or irinotecan or other drug comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form.

The compositions of the invention can comprise excipients that are well known in the art and are listed, for example, in the U.S. Pharmacopeia (USP) 25-NF20 (2002), which is incorporated herein in relevant part. In general, compositions of the invention comprise one or more active ingredients, a binder/filler, and a lubricant in pharmaceutically compatible and pharmaceutically acceptable amounts. In certain embodiment, dosage forms comprise sodium metal arsenite and optionally one or more other active ingredients, microcrystalline cellulose, pre-gelatinized starch, and magnesium stearate.

The dosage and treatment regimens and routes of administration of sodium meta arsenite and irinotecan discussed above may also be applied to the treatment of rectal cancer in patients in need of such treatment.

The dosage and treatment regimens and routes of administration of sodium meta arsenite and irinotecan discussed above may also be applied to the treatment of other solid tumors by replacing the irinotecan with any acceptable drug that is efficacious in the treatment of the particular tumor, particularly drugs that have a different mechanism of action from intraperitoneally administered sodium meta arsenite. For example, intraperitoneal administration of a therapeutically effective amount of sodium meta arsenite can be used in combination with the administration of an anti-VEGF cancer agent such as bevacizumab or anti-VEGF antibody, or radiation therapy to treat various solid tumors. The skilled practitioner can select appropriate anti-cancer agents for a particular tumor type to be used in combination with intraperitoneal administration of sodium meta arsenite to obtain the beneficial and enhanced effects of the combination.

### Example 1

### Observation of Necrosis in Tumor Tissues after Treatment with Sodium Meta Arsenite

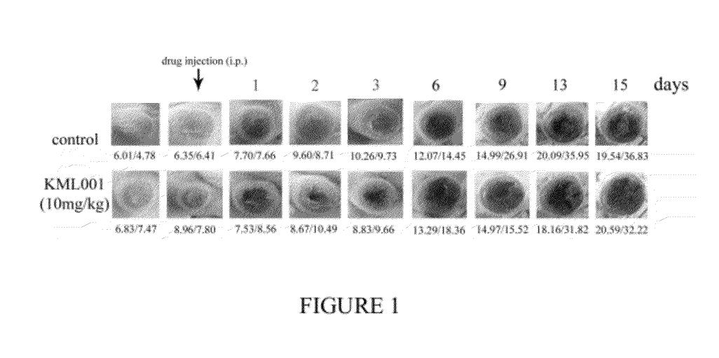

Seven-week-old, female, specific-pathogen-free (BALB/c) white mice (weight: approximately 20 g) purchased from ORIENTBIO Inc. (Seoul Korea) were subcutaneously injected with 2×10⁶ of CT26 colon carcinoma cells per mouse. When the tumor size became 3 mm in diameter, the control group was injected with a phosphate buffered saline (PBS) solution with 5% dextrose while the experimental group was intraperitoneally injected with 100 μL of sodium meta arsenite at a concentration of 10 mg/kg with 5% dextrose. After injection, the measurements of the size of tumors (volume=πab²/6, where a and b are diameters of tumors) by using the caliper once every two days and the images of the tumors were recorded for 15 days. The results are shown in FIG. 1. While the discoloration of the tissues of the mice injected without sodium meta arsenite was not observed for a certain period of time, the central part of tumors in the tissues of the white mice injected with sodium meta arsenite became discolored at 24 hours after the injection of sodium meta arsenite. As time went by, it was observed that the tumors became discolored regardless of the injection of sodium meta arsenite. In case of white mice injected with sodium meta arsenite, the tissues in the central part became discolored due to necrosis.

### Example 2

### Histologic Analysis of Tumor Tissues Treated with Sodium Meta Arsenite

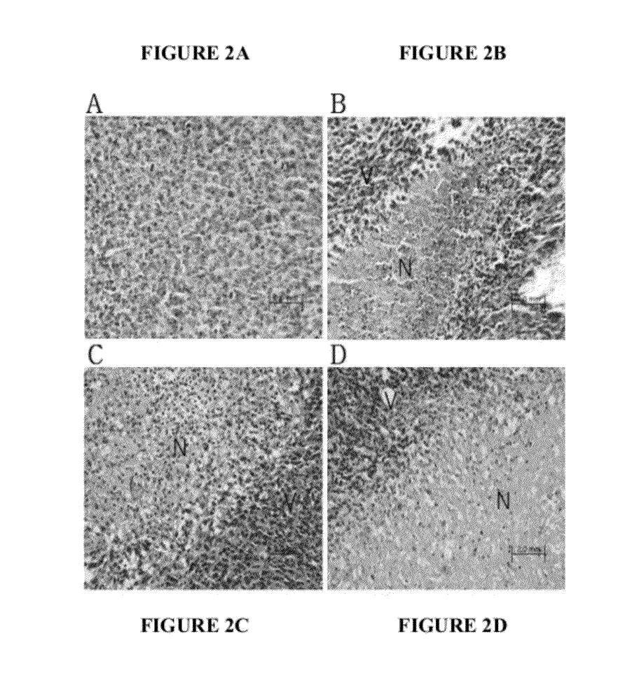

Seven-week-old, female, specific-pathogen-free (BALB/c) white mice (weight: approximately 20 g) purchased from ORIENTBIO Inc. (Seoul Korea) were subcutaneously injected with 2×10⁶ cells of the CT26 colon carcinoma cell line per mouse. When the tumor size reached 3 mm in diameter, the control group was injected with a phosphate buffered saline (PBS) solution with 5% dextrose while the experimental group was intraperitoneally injected with 100 μL of KML001 at a concentration of 10 mg/kg with 5% dextrose. At 8, 24 and 48 hours after injection, liver, spleen and tumor tissue samples were taken and placed into a 37% solution of formaldehyde for 24 hours. The tissues were inserted into paraffin and sectioned at 4 μm on a microtome (made by SLEE MAINZ GmbH in Germany) and the sections were placed on slides. The sections were stained with hematoxylin (using Mayer's hematoxylin solution, Sigma, MHS-16) and eosin (using Eosin Y solution, Sigma) dyes and the images were observed with an Olympus inverted microscope (Bx 50). The results are shown in FIG. 2. Tumor tissues obtained from mice treated with control (no sodium meta arsenite) showed no change in cell density and no changes in cell distribution or shape of the cells were observed. In contrast, with increasing exposure time to sodium meta arsenite, the scope of necrotic cell death in the tumor tissues of the mice injected with sodium meta arsenite was observed to increase and it was observed that necrotic cell death was caused from the central part of the tumors. No ischemic damage was observed in liver, spleen or kidney tissues in the sodium meta arsenite-treated mice.

### Example 3

### Analysis of Apoptosis and Cytotoxicity of HUVECs by KML001

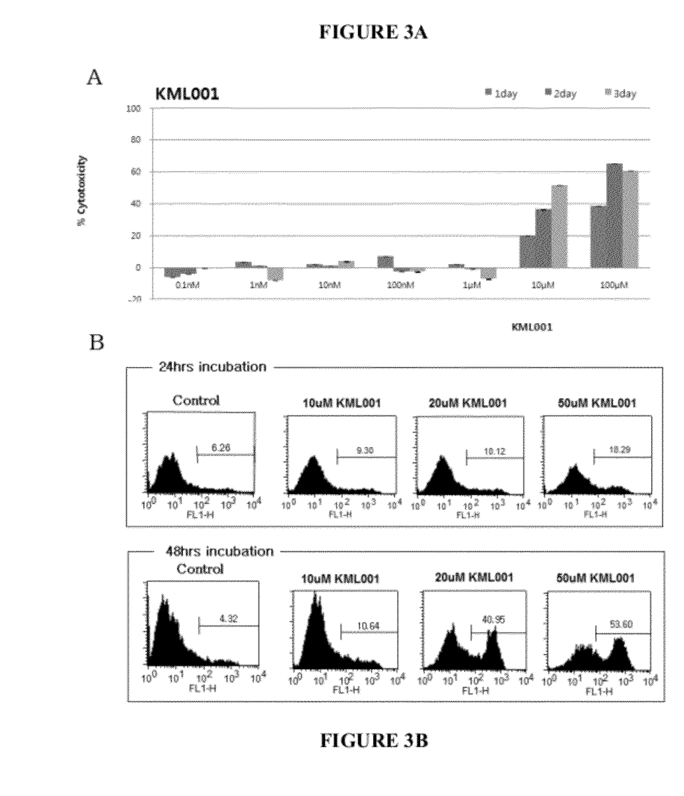

To determine the effect, if any, of sodium meta arsenite on vascularization, the effect on endothelial cells (which are essential for vascularization) was first analyzed. Human umbilical cord vein endothelial cells (HUVECs offered by ATCC) were cultivated in 96-well microculture plates containing F-12K (Sigma) medium supplemented with 10% FBS and endothelial cell growth supplement (ECGS, E2759, Sigma). For the cytotoxicity test of the cells after 24 hour cultivation, the cells were treated with KML001 at the concentration of 0.1 nM, 1 nM, 10 nM, 1 uM, 10 uM and 100 uM. At 24 hours and 48 hours after treatment, 20 μl/well of CellTiter 96® Aqueous One Solution (by Promega) Reagent was added on each well and reacted for one hour at 37° C. The reacted 96-well plates were measured at 490 nm using a spectrophotometer (UVStar, Greiner Bio-One). To analyze apoptosis of HUVECs, the cells were cultivated on F25 flasks using the same method as discussed above and then the cells were treated with sodium meta arsenite at a concentration of 10 uM, 20 uM and 50 uM. Cells were collected after 24 and 48 hours. The collected cells were stained for 30 minutes with Annexin-V for FACS analysis. After two washes, the cells were analyzed by using flow cytometry (FACSCALIBUR, BECTON DICLINSON). As shown in A of FIG. 3, cytotoxicity was observed at 10 uM and the cytotoxicity of sodium meta arsenite was significantly increased over time. In addition, (sodium meta arsenite is known to cause a variety of physiologic effects. As shown in FIG. 3B, FACS analysis showed that apoptosis caused by sodium meta arsenite is concentration dependent, increasing with increased concentration. Apoptosis also increased over time with exposure to sodium meta arsenite.

### Example 4

### Change in Microtubules from HUVECs Treated with Sodium Meta Arsenite

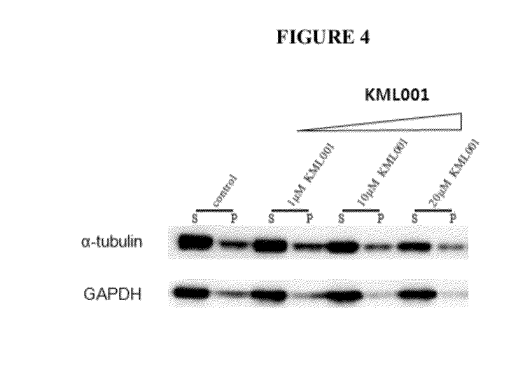

To investigate whether such apoptosis phenomenon is caused by a cytoskeletal abnormality that affects cell division and cell differentiation, the change in α-tubulin expression in the cytoskeleton was identified by the division of tubulin into isolated form (soluble, S) and polymerized form (ppt, P). HUVECs (obtained from ATCC) were cultivated in F25 culture flasks containing F-12K (Sigma) medium supplemented with 10% FBS and endothelial cell growth supplement (ECGS, E2759, Sigma). After cultivation for 24 hours, the cells were treated with sodium meta arsenite at a concentration of 1 uM, 10 uM and 20 uM. Cells were collected 24 hours after treatment. The collected cells were suspended in RIPA buffer (50 mM Tris, pH 7.5, 150 mM NaCl, 1% NP-40, 0.5% sodium deoxychloate, 0.1% SDS, protease inhibitor) at room temperature for 10 minutes and then centrifuged for 30 minutes at 22° C. at 13,200 rpm to separate the supernatant liquid from the sediment. RIPA solution was added to the sediment in the same amount and the sediment was crushed using an ultrasonic cell crusher. The protein from the supernatant liquid and the sediment were assayed using a BCA protein kit (Thermo, 23227) and then the same amount of protein (3 μg) was mixed with protein sample buffer and it was warmed up in a water bath in boiling water at 100° C. for five minutes. SDS-PAGE was conducted on a 10% gel. At the end of the electrophoresis, the gel was transferred to nitrocellulose membrane in a semi-dry method at the room temperature at 220 mA for 40 minutes. The nitrocellulose membrane was reacted with tris-buffered saline (TBS-T) containing 5% skim milk for one hour to prevent non-specific antibody binding. Monoclonal antibodies against α-tubulin (SantaCruze, sc-32293) and GAPDH (SantaCruze, sc-47724) were diluted with tris-buffered saline (TBS-T) containing 0.1% tween and they were reacted at room temperature for one hour. After washing with TBS-T three times, the membrane was reacted with Goat anti-Mouse lgG secondary antibody (SantaCruze, sc-2005) conjugated to HRP at the room temperature for one hour and then exposed to light with ChemiDoc (Bio-Rad Molecular Imager ChemiDoc XRS) by using Immun-Star™ WesternC™ Chemiluminescent kit (Bio-Rad, 170-5070) to analyze. As shown in FIG. 4, the higher concentration of sodium meta arsenite reduced the protein level on both the supernatant liquid (isolated tubulin) and the sediment (polymerized tubulin). This means that sodium meta arsenite not only isolates the polymerized tubulin from the polymer, but also reduces the overall amount of tubulin by inducing the isolated tubulin protein breakdown.

### Example 5

### Change in tubulin protein from HUVECs treated with KML001

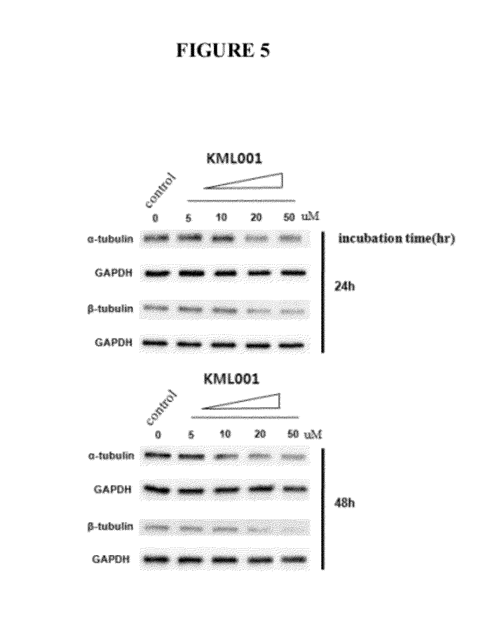

To analyze the change in the overall expression of tubulin, the total amount of tubulin protein was kept to identify by western blotting. HUVECs were cultured in F25 culture flasks containing F-12K (Sigma) medium supplemented with 10% FBS and endothelial cell growth supplement (ECGS, E2759, Sigma). After cultivation for 24 hours, the cells were treated with sodium meta arsenite at concentrations of 5 uM, 10 uM, 20 uM and 50 uM. At 24 hours and 48 hours after treatment, the cells were collected and washed with phosphate buffered saline (PBS) solution. The washed cells were reacted with THF solution (50 mM Tris-Hcl, pH 7.5, 100 mM NaCl, 2 mM EDTA, 1% NP-40, protease inhibitor) at 4° C. for 15 minutes and then broken down or destroyed by use of an ultrasonic cell crusher before SDS (0.5%) was added. Protein from the samples was assayed by using a BCA protein kit (Thermo, 23227) before the same amount of protein (3 μg) was mixed with protein sample buffer. The protein (3 μg) was mixed with protein sample buffer and warmed up in a water bath in boiling water at 100° C. for five minutes. SDS-PAGE was conducted using a 10% polyacrylamide gel. At the end of electrophoresis, the gel was transferred to nitrocellulose membrane in semi-dry method at room temperature at 220 mA for 40 minutes. The nitrocellulose membrane was reacted with tris-buffered saline (TBS-T) containing 5% skim milk for one hour to prevent non-specific antibody binding. Monoclonal antibodies against α-tubulin (SantaCruze, sc-32293), β-tubulin (SantaCruze, sc-58883) and GAPDH (SantaCruze, sc-47724) were diluted with tris-buffered saline (TBS-T) containing 0.1% tween and reacted at the room temperature for one hour. After the membranes were washed three times with TBS-T for five minutes, the membranes were reacted with Goat anti-Mouse lgG secondary antibody (SantaCruze, sc-2005) conjugated to HRP at room temperature for one hour and then exposed to light with ChemiDoc (Bio-Rad Molecular Imager ChemiDoc XRS) by using Immun-Star™ WesternC™ Chemiluminescent kit (Bio-Rad, 170-5070) to analyze. As shown in FIG. 5, the higher concentration of sodium meta arsenite reduced the protein level of α-tubulin and β-tubulin, indicating that sodium meta arsenite specifically destroys α-tubulin and β-tubulin and then reduces the amount of protein.

### Example 6

### Change in Tubulin mRNA from HUVECs Treated with Sodium Meta Arsenite

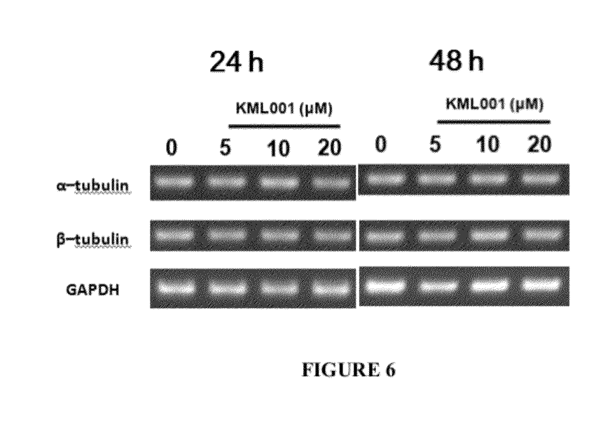

To determine whether the level of tubulin gene expression is responsible for the change in amount of tubulin protein observed in Example 5, RT-PCR was performed. HUVECs (obtained from ATCC) were cultivated in F25 culture flasks containing F-12K (Sigma) medium supplemented with 10% FBS and endothelial cell growth supplement (ECGS, E2759, Sigma). After cultivation for 24 hours, the cells were treated with sodium meta arsenite at concentrations of 5 uM, 10 uM and 20 uM. Cells were collected at 6 hours and 48 hours after treatment. Total RNA was extracted using Trizol (Invitrogen, USA) and alcohol precipitation. 1 ug of total RNA was used for reverse transcriptase and dT was used for cDNA synthesis. The primer for synthesized cDNA and α-tubulin (sense: 5′-ATT GTG CCT TCA TGG TAG AC-3′ (SEQ ID NO. 1), antisense: 5′-TTC TGT CAG GTC AAC ATT CA-3′) (SEQ ID NO. 2), the primer for β-tubulin (sense: 5′-AAC GAC CTC GTC TCT GAG TA-3′ (SEQ ID NO. 3), antisense: 5′-AAT TCT GAG GGA GAG GAA AG-3′) (SEQ ID NO. 4) and GAPDH primer set (sense: 5′-ACC ACT TTG TCA AGC TCA TT-3′ (SEQ ID NO. 5), antisense: 5′-AGT GAG GGT CTC TCT CTT CC-3′) (SEQ ID No. 6) were used for denaturation at 95° C. for four minutes. After the reactions at 23 cycles at 95° C. for 30 seconds, at 52° C. for 30 seconds and at 72° C. for 40 seconds, PCR was performed at 72° C. for four minutes. The PCR product was confirmed by 1.5% agarose gel electrophoresis. As shown in FIG. 6, α-tubulin and 13-tubulin mRNA levels had no change at 24 hours and 48 hours after treatment with sodium meta arsenite at each concentration. The result clearly demonstrates that the quantitative change in amount of α-tubulin and β-tubulin in HUVECs was not caused by a change in the expression of mRNA, but instead was caused by a change in the protein level.

### Example 7

### Analysis of Change in Microtubules by Using Immunohistochemistry from HUVECs Treated with KML001

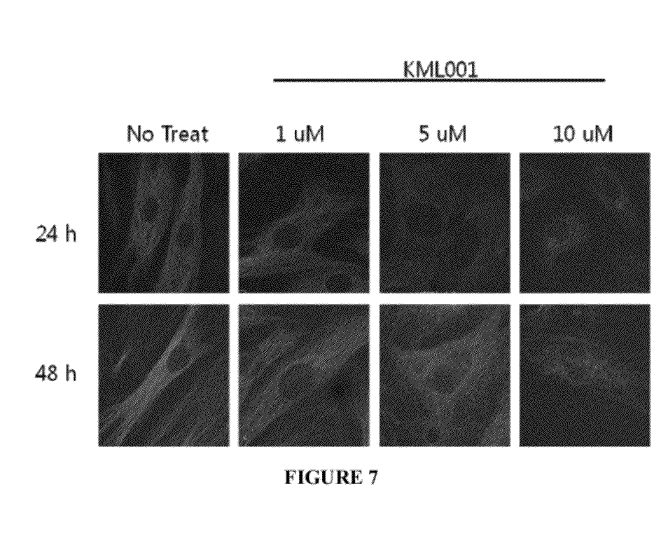

HUVECs were cultured in a confocal dish (made by SPL, Korea) with F-12K (Sigma) medium containing 10% FBS and endothelial cell growth supplement (ECGS, E2759, Sigma). After cultivation for 24 hours, the cells were treated with sodium meta arsenite at a concentration of 1 uM, 5 uM and 10 uM. At 24 hours and 48 hours after treatment, the cells were fixed with 3.7% paraformaldehyde at room temperature for 10 minutes. After washing with phosphate buffered saline (PBS) solution three times, the cells were treated with 0.15% Triton X-100 for 15 minutes and then washed three times with PBS solution. After blocking with 2% bovine serum albumin (BSA, Sigma) at room temperature for 60 minutes, the cells were washed three times with PBS solution. Monoclonal antibody against α-tubulin (SantaCruze, sc-32293) was reacted with the cells at room temperature for one hour and then the cells were washed three times with the PBS solution. The cells were then reacted with rat anti-mouse IgG1 secondary antibody (Pharmigen, #A85-1) conjugated to FITC. After being washed three times with PBS solution, they were analyzed using a confocal laser scanning microscope (OLYMPUS FV500,1×81). As shown in FIG. 7, the cells treated with sodium meta arsenite showed a reduction in the total amount of microtubules, which was concentration dependent.

### Example 8

### Analysis of the Anti-Tumor Effects of Irinotecan and Sodium Meta Arsenite

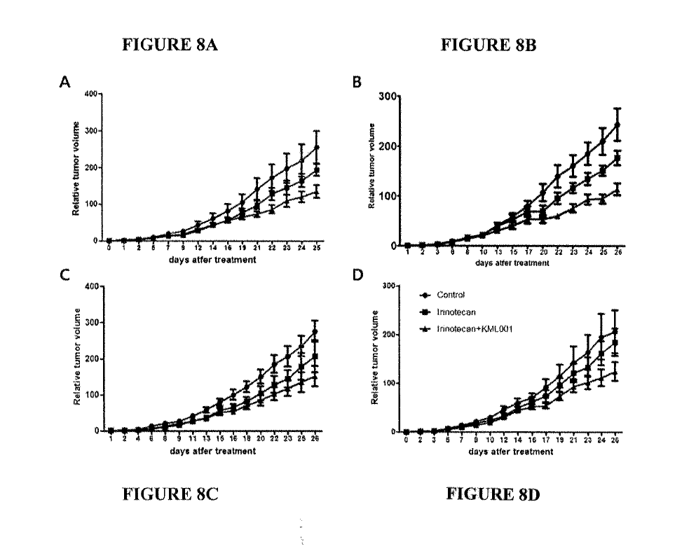

Irinotecan and sodium meta arsenite were tested and observed for their anti-tumor effects for 7 weeks in female mice (BALB/c, ˜20 g) using various delayed injection schedules. Colon cancer cells CT26 were injected subcutaneously into female mice at 2×10⁶ cancer cells, resulting in a 3 mm tumor. Irinotecan was initially injected at 100 ul at a dosage of 10 mg/kg. Following this initial injection, 15 mg/kg sodium meta arsenite was intraperitoneally injected at 24 hours after (FIG. 8A), 24 hours and 72 hours after (FIG. 8B), 72 hours after (FIG. 8C), or 30 minutes after (FIG. 8D) the initial injection. The results are shown in FIG. 8.

### Example 9

Female BALBc mice bearing subcutaneous CT26 colon carcinoma cells were injected intraperitoneally with sodium meta arsenite (10/mg/kg body weight) or irinotecan alone, or co-administered weekly for four weeks. Massive necrosis in the central part of the tumor was observed by 24 hours after treatment with sodium meta arsenite alone and regrowth was observed from the periphery after two days. Significant tumor growth delay was observed when sodium meta arsenite was administered after 24 hours or 72 hours after irinotecan.

### Example 10

When KML001 was injected after tumors were generated through cell experiments and animal experiments, there was no effect on the reduction in tumor volume but it was found that apoptosis occurred dose-dependently and necrotic phenomena in tumor tissues occurred. To determine whether the occurrence of central necrosis of tumors 24 hours after treatment was caused by the reduction of vascularity, DCE-MRI was used before the medication was administered and 24 hours after administration.

Experiment Schedule

1st: Drug KML001 injection—3 mice

2nd: Drug KML001 injection—5 mice

3rd: Arsenic trioxide (ATO) injection to a control group—5 mice on

4th: Arsenic trioxide (ATO) injection to a control group—4 mice

5th: Saline injection to a control group—4 mice

| | | | | | |
|---|---|---|---|---|---|
| | Group 1 | | Group 3 | Group 4 | Group 5 |
| Days | (n = 3) | Group 2 (n = 5) | (n = 5) | (n = 4) | (n = 4) |

| 0 | Cancer cell injection | | |
|---|---|---|---|
| 10 | Baseline MRI and KML001 | Baseline MRI and | Baseline MRI and |
| | injection | ATO injection | Saline injection |

| | |
|---|---|
| 11 | Post Treatment MRI |

Preparation of Animal Models

BALB/C Female Mice of 7 Weeks

Inoculate CT26 (mouse colon cancer) 2×10⁶ cell subcutaneously into mice on the upper parts of their thighs.

After checking the tumor size between 6 to 10 mm, acquire images through MRI.

Image Acquisition: Use a magnetic resonance imaging device (i.e., 3 Tesla Intera human clinical scanner; Philips medical systems) and a mouse coil with an inner diameter of 4 cm to get images. Acquire images before the drug is administered and those 24 hours after that for two days.

MR Pulse Sequence as follows: Dynamic Contrast enhanced MRI scan: FOV (mm)=50×35; RFOV (%)=71; Matrix scan=112; reconstruction=224; TR/TE (ms)=12/4.0; slice thickness=1 mm; slice number=11; dynamic scans=60.

MRI Imaging:

i. Conduct an experiment to the subjects under anesthesia by using an inhalation anesthetic mixing 1.5% isoflurane in 100% oxygen at a flow rate of 1 L/min.

ii. Cannulate an intravenous injection line through the tail vein to inject a contrast agent.

iii. Use Gd-DTPA (Magnevist, Schering, Germany, 281 mg/kg) as a contrast agent and get pre-contrast images before injecting the contrast agent and post-contrast images after injecting it.

iv. After getting the images, inject KML001 (10 mg/kg) and Arsenic trioxide (7.5 mg/kg) intraperitoneally.

v. Get images through a dynamic scan again 24 hours after injection.

Results

Experimental animals whose dynamic contrast-enhanced images failed to be acquired due to advance enhancement or nude mice whose intravenous injection lines failed to be cannulated were excluded from the results. Accordingly, the groups of animals with which the DCE-MRI data was obtained are as follows: Group of animals treated with ATO: n=4, Group of animals treated with Saline: n=4 and Group of animals treated with KML001: n=6.

As the low data in MRI was mapped from each result of the DCE-MRI to the positive control group of animals treated with ATO, the negative control group of animals treated with saline and the experimental group of animals treated with KML001, Kep values important for tumor vascularity were calculated. Kep (rate constant) is the back diffusion rate constant from interstitial space to vascular space, reflecting leakage of tumor vessels, i.e., a measurement of vascularity.

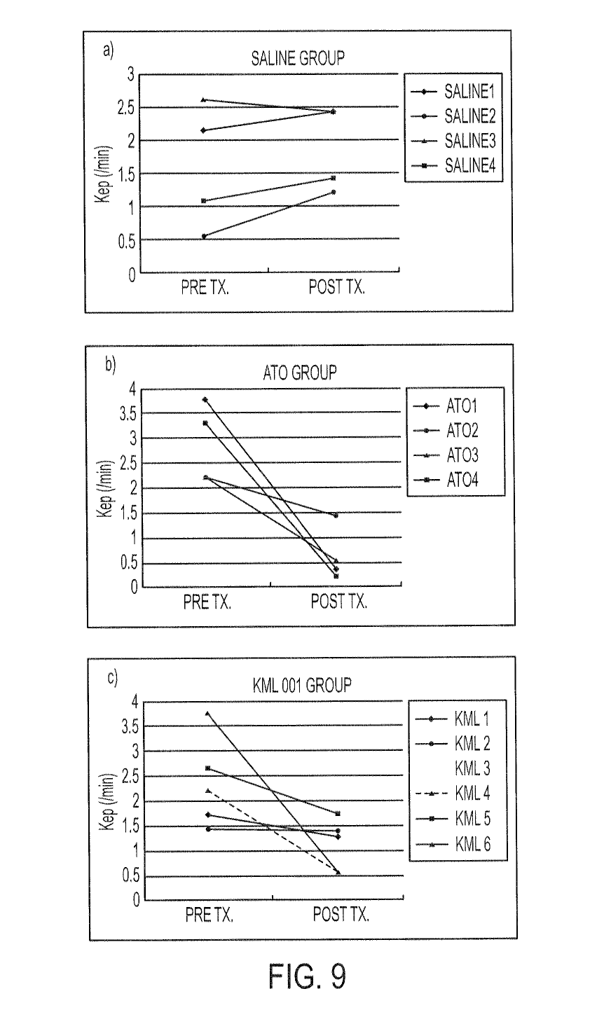

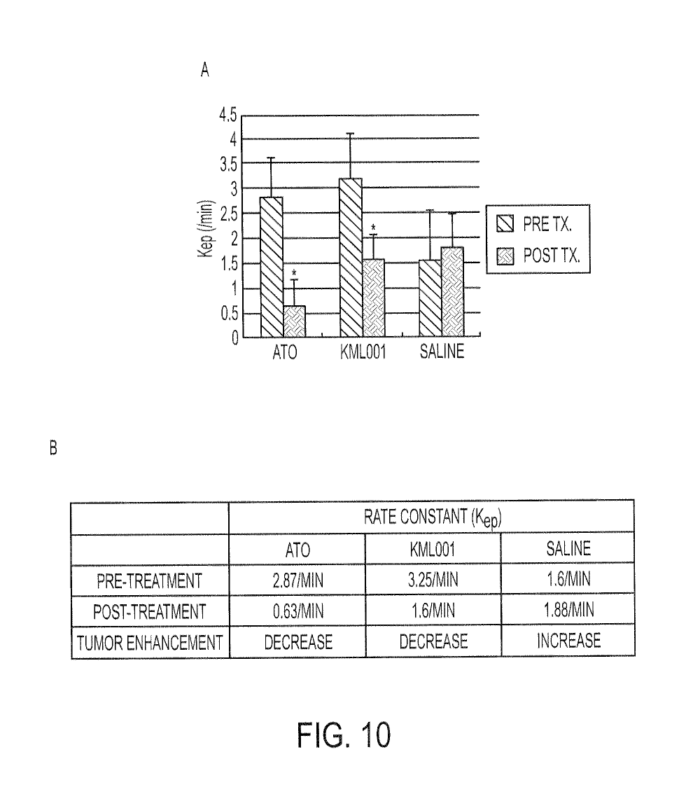

As shown in the graphs in FIG. 9, Kep values in the negative control group of animals treated with saline were not significantly changed or increased. But Kep values of both groups of animals treated with ATO and KML001 were significantly reduced (p<0.05: ATO=0.004, KML001=0.032, Saline=0.659). This means that vascularity in both groups is reduced after treatment in comparison of the group of animals treated with the conventional anticancer agent ATO and the group of animals treated with a new agent KML001 (FIG. 10).

### CONCLUSION

Kep values were reduced after the treatment with ATO and KML001 and it was confirmed that the contrast enhancement of the tumors was decreased in the MRI 24 hours after treatment.

What is claimed is:

1. A method of treating a solid tumor in a patient in need thereof comprising intraperitoneally administering to the patient a therapeutically effective amount of sodium meta arsenite and an anti-cancer agent that has a different mechanism of action from intraperitoneally administered sodium meta arsenite.

2. The method of claim 1 wherein the sodium meta arsenite is administered once a week.

3. The method of claim 1 wherein the anti-cancer agent is an anti-VEGF agent.

4. The method of claim 1 wherein the anti-cancer agent is irinotecan.

5. The method of claim 4 wherein the solid tumor is colon cancer.

6. The method of claim 5 wherein the solid tumor is colorectal cancer.

7. The method of claim 1 wherein the sodium meta arsenite and anti-cancer agent are administered separately.

8. The method of claim 7 wherein the sodium meta arsenite is administered to the patient within 24 to 72 hours of administration of the anti-cancer agent to the patient.

9. The method of claim 4 further comprising administering radiation to the patient prior to, after or during a treatment regimen with sodium meta arsenite and/or irinotecan.

10. A method for treating colon tumor(s) in a subject comprising: (a) intraperitoneally administering to a subject in need thereof a combination of one or more therapeutic doses of sodium meta arsenite and one or more therapeutic doses of irinotecan; (b) monitoring the colon cancer cells and/or the bulk colon cancer tumor size and/or integrity in the subject prior to and/or after administration of a predetermined number of doses of the sodium meta arsenite and/or the irinotecan; and (c) determining whether there is a reduction in the amount of viable colon cancer cells and/or the bulk colon tumor size or integrity of the tumor; and (d) repeating steps (a), (b) and (c) as necessary.

11. The method of claim 10 wherein the sodium meta arsenite and irinotecan are administered separately.

12. The method of claim 10 wherein the sodium meta arsenite is administered intermittently.

13. The method of claim 12 wherein the sodium meta arsenite is administered on a weekly basis.

14. A kit for the treatment of a solid tumor comprising one or more dosage units of sodium meta arsenite formulated for intraperitonal injection and one of more dosage units of irinotecan.


SEQUENCE LISTING (6 sequences)

SEQ ID NO: 1 - DNA, 20 bp, Artificial Sequence
  primer for synthesized cDNA and a-tubulin chemically synthesized
attgtgccttcatggtagac

SEQ ID NO: 2 - DNA, 20 bp, Artificial Sequence
  antisense primer for synthesized cDNA and a-tubulin chemically synthesized
ttctgtcaggtcaacattca

SEQ ID NO: 3 - DNA, 20 bp, Artificial Sequence
  primer fo b-tubulin chemically synthesized
aacgacctcgtctctgagta

SEQ ID NO: 4 - DNA, 20 bp, Artificial Sequence
  antisense primer for b-tubulin chemically synthesized
aattctgagggagaggaaag

SEQ ID NO: 5 - DNA, 20 bp, Artificial Sequence
  glyceraldehyde-3-phosphate dehydrogenase (GAPDH) primer chemically synthesized
accactttgtcaagctcatt

SEQ ID NO: 6 - DNA, 20 bp, Artificial Sequence
  glyceraldehyde-3-phosphate dehydrogenase (GAPDH) antisense primer chemically synthesized
agtgagggtctctctcttcc